(19) United States
(12) Patent Application Publication
(10) Pub. No.: US 2007/0253926 A1
(43) Pub. Date: Nov. 1, 2007

(54) Packaged cleaning composition concentrate and method and system for forming a cleaning composition

(76) Inventors: Tami J. Tadrowski, Greensboro, NC (US); Leighton Davies-Smith, Oak Ridge, NC (US); Kim R. Smith, Woodbury, MN (US); Mark D. Levitt, St. Paul, MN (US); Stephen Engel, Mounds View, MN (US); David J. Falbaum, Minneapolis, MN (US); Jerry D. Hoyt, Hastings, MN (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 11/413,275

(22) Filed: Apr. 28, 2006

(51) Int. Cl. A61K 8/81; A61K 8/73; C11D 3/20

(52) U.S. Cl. 424/70.13; 424/70.16; 510/180

(57) ABSTRACT

A packaged cleaning composition concentrate includes a container for holding a cleaning composition concentrate, and a cleaning composition concentrate. The cleaning composition concentrate has a solids content of at least about 1 wt. % based on the weight of the cleaning composition concentrate. The cleaning composition concentrate includes a surfactant component, a dispersant component, and at least one of a sheeting agent or a humectant. A method for forming a use composition is provided.

### FIELD OF THE INVENTION

The invention relates to a packaged cleaning composition concentrate, and to a method for forming a cleaning composition. The cleaning composition can be provided in the form of a concentrate on a substrate, or in the form of a concentrate free from a substrate. The cleaning composition concentrate, with or without substrate, can be provided in a container. The cleaning composition concentrate can be combined with water to provide a use composition for use in cleaning hard surfaces such as glass, tile, countertops, etc. The cleaning composition can tolerate water that can be considered hard water.

### BACKGROUND OF THE INVENTION

Glass cleaners are often available in a form that is ready to use. A consumer can purchase a glass cleaner, such as, a window cleaner, and use the glass cleaner directly on a glass surface. One reason that glass cleaners are provided in a form that is ready to use is to control the presence of water hardness in the ready to use glass cleaner. Water hardness has a tendency to cause precipitation of anionic surfactant. Because glass cleaners contain a large percentage of water, deionized water is often used to formulate glass cleaners in order to avoid precipitation of anionic surfactants present in the glass cleaners.

Exemplary disclosures of glass cleaner compositions include U.S. Pat. No. 6,420,326 to Maile et al., U.S. Pat. No. 5,534,198 to Masters et al., U.S. Pat. No. 5,750,482 to Cummings, U.S. Pat. No. 5,798,324 to Svoboda, and U.S. Pat. No. 5,849,681 to Newmiller.

### SUMMARY OF THE INVENTION

A packaged cleaning composition concentrate is provided according to the invention. The package cleaning composition concentrate includes a container for holding a cleaning composition concentrate and a cleaning composition concentrate. The cleaning composition concentrate has a solids content of at least about 1 wt. % based on the weight of the cleaning composition concentrate. The cleaning composition concentrate includes a surfactant component, a dispersant component, and at least one of a sheeting agent or a humectant.

Methods for forming a cleaning composition are provided according to the invention. The methods generally include combining a cleaning composition concentrate with water of dilution to form a use composition. If the water of dilution is hard water, the cleaning composition concentrate can be provided to handle the hardness in the water. In one method, a packaged cleaning composition can be provided wherein the container that contains the cleaning composition concentrate can be provided as a water soluble or water dispersible film. In an alternative embodiment, a multiple reservoir cartridge can be placed in the neck of a spray bottle wherein the multiple reservoir cartridge contains multiple reservoirs each containing a cleaning composition concentrate, and the method can include puncturing one of the reservoirs so that the cleaning composition concentrate combines with water of dilution in the spray bottle. In an alternative method, the cleaning composition concentrate can be provided on a substrate, and the substrate containing the cleaning composition concentrate can be combined with water of dilution. The substrate containing the cleaning composition concentrate can be provided in a spray bottle. The substrate can be provided as a sleeve for sliding over a spray bottle dip tube. An alternative method for performing a cleaning composition can include combining a cleaning composition concentrate with water of dilution at a weight ratio of the concentrate to the water of dilution of about 1:1 to about 1:1000 and can be provided as a batch operation or as a continuous operation.

### BRIEF DESCRIPTION OF THE DRAWINGS

FIG. 1 is a perspective view of a packaged cleaning composition concentrate according to the principles of the present invention.

FIG. 2 is a front view of a packaged cleaning composition concentrate according to the principles of the present invention.

FIG. 3 is a front view of a packaged cleaning composition concentrate provided on a substrate according to the principles of the present invention.

FIG. 4 is a cross-sectional view of the sleeve shown in FIG. 3 taken along lines 4-4.

FIGS. 5(a)-(c) are cross-sectional views of exemplary sleeves for use on a dip tube according to the principles of the present invention.

FIG. 6 is a perspective view of a container for holding multiple doses of cleaning composition concentrate according to the principles of the present invention.

FIG. 7 is a schematic view of an apparatus for dispensing a cleaning composition according to the principles of the present invention.

FIG. 8 is a schematic view of an apparatus for dispensing a cleaning composition according to the principles of the present invention.

### DETAILED DESCRIPTION OF THE INVENTION

The cleaning composition can be referred to as a detergent composition and can be provided in the form of a concentrated detergent composition or as a ready to use detergent composition. The concentrated detergent composition can be referred to as the concentrate, and can be diluted to provide the ready to use detergent composition or the use composition. The ready to use detergent composition can be referred to as the use composition when it is the composition that is intended to be used to provide cleaning of a surface. In addition, the ready to use detergent composition can be further diluted to provide the use composition that is intended to be used to clean a surface. In the case of a glass cleaner, the ready to use composition can be the use composition and can be applied directly to a surface without further dilution. When cleaning certain hard surfaces, such as a counter or a floor, it may be desirable to dilute the ready to use composition (e.g., by placing a portion of the ready to use composition into a bucket of water) and clean the hard surface with the resulting use composition.

The cleaning composition can be provided as a concentrate for shipment to retail distributors, commercial end users, or non-commercial end users. The retail distributors or the commercial end users can dilute the concentrate to provide a less concentrated detergent composition or a ready to use detergent composition. The retail distributors can package and sell the less concentrated detergent composition or the ready to use detergent composition to consumers. In the case of a glass cleaner, the retail distributor can dilute the concentrate to provide a glass cleaner in a ready to use form, and then package the glass cleaner for sale to consumers. Commercial end users, such as, car washing facilities and janitorial services, can dilute the concentrate to achieve a ready to use composition and then use the ready to use composition in their cleaning service. Non-commercial end users can purchase the concentrate and form the ready to use composition or can purchase the ready to use composition.

By providing the cleaning composition as a concentrate, the concentrate can be diluted with the water available at the locale or site of dilution. It is recognized that the level of water hardness can change from one locale to another. Accordingly, the concentrate can be formulated so that it can be diluted with water having varying amounts of hardness depending upon the locale or site of dilution while providing a desirable ready to use composition or use composition.

In general, water hardness refers to the presence of calcium, magnesium, iron, manganese, and other polyvalent metal cations that may be present in the water, and it is understood that the level of water hardness can vary from municipality to municipality. Because of the likely fluctuation in water hardness levels, concentrated detergent composition can be formulated to handle differing water hardness levels found at varying locations without having to soften the water or remove the hardness from the water. High solids containing water can be considered to be water having a total dissolved solids (TDS) content in excess of 200 ppm. In certain localities, the service water can contain a total dissolved solids content in excess of 400 ppm, and even in excess of 800 ppm. Water hardness can be characterized by the unit “grain” where one grain water hardness is equivalent to 17.1 ppm hardness expressed as CaCO₃. Hard water can be characterized as water having at least 1 grain hardness. Hard water is commonly available having at least 5 grains hardness, at least 10 grains hardness, or at least 20 grains hardness.

The hardness in water can cause anionic surfactants to precipitate. Visual precipitation refers to precipitate formation that can be observed by the naked eye without visual magnification or enhancement. In order to protect the anionic surfactant component in the cleaning composition of the invention, a water hardness anti-precipitant mixture can be provided that includes a dispersant and at least one of a sheeting agent or a humectant. The cleaning composition can include additional surfactants and other components commonly found in cleaning compositions.

Now referring to FIG. 1, a packaged cleaning composition concentrate is shown at reference number 10. The packaged cleaning composition concentrate 10 includes a film 12 and a cleaning composition concentrate 14 provided within the film 12. The film 12 can be a water soluble film or a non-water soluble film. In the case of a non-water soluble film, the film can be torn or cut to release the cleaning composition concentrate 14. The cleaning composition concentrate 14 can then be introduced into a volume of water. For example, the cleaning composition 14 can be poured into a container and combined with water. When the film 12 is a water soluble film (or a water-dispersible film), the packaged cleaning composition concentrate 10 can be introduced into a volume of water and, with time, the film 12 dissolves, disintegrates, or disperses, and the cleaning composition concentrate 14 contacts the water.

The film 12 can be provided so that it encloses or contains the cleaning composition concentrate 14. The film 12 can be provided having a fold line 16, and can be provided having a heat seal or adhesive seal along the edges 18. It should be understood that the packaged cleaning concentrate 10 can be provided without a fold line and the edges can all be heat sealed or adhesively sealed.

Non-water soluble films that can be used to hold or contain the cleaning composition concentrate include conventional films used in the packaging industry. Exemplary films that can be used include polyethylenes, polypropylenes, polybutylenes, polyesters, and polyamides.

Water solible (or water dispersible) films that can be used include those made from water soluble polymers such as those described in Davidson and Sittig, Water Soluble Resins, Van Nostrand Reinhold Company, New York (1968), herein incorporated by reference. The water soluble polymers can have proper characteristics such as strength and pliability in order to permit machine handling. Exemplary water soluble polymers include polyvinyl alcohol, cellulose ethers, polyethylene oxide, starch, polyvinylpyrrolidone, polyacrylamide, polyvinyl methyl ether-maleic anhydride, polymaleic anhydride, styrene maleic anhydride, hydroxyethylcellulose, methylcellulose, polyethylene glycols, carboxymethylcellulose, polyacrylic acid salts, alginates, acrylamide copolymers, guar gum, casein, ethylene-maleic anhydride resin series, polyethyleneimine, ethyl hydroxyethylcellulose, ethyl methylcellulose, and hydroxyethyl methylcellulose. Lower molecular weight water soluble, polyvinyl alcohol film-forming polymers are generally, preferred. Polyvinyl alcohols that can be used include those having a weight average molecular weight of between about 1,000 and about 300,000, and between about 2,000 and about 150,000, and between about 3,000 and about 100,000.

Exemplary water soluble packaging films are disclosed in U.S. Pat. Nos. 6,503,879; 6,228,825; 6,303,553; 6,475,977; and 6,632,785, the disclosures of which are incorporated herein by reference. In addition, see U.S. Pat. No. 4,474,976 to Yang, U.S. Pat. No. 4,692,494 to Sonenstein, U.S. Pat. No. 4,608,187 to Chang, U.S. Pat. No. 4,416,793 to Haq, U.S. Pat. No. 4,348,293 to Clarke, U.S. Pat. No. 4,289,815 to Lee, and U.S. Pat. No. 3,695,989 to Albert, the disclosures of which are incorporated herein by reference. An exemplary water soluble polymer that can be used to package the concentrate includes polyvinyl alcohol.

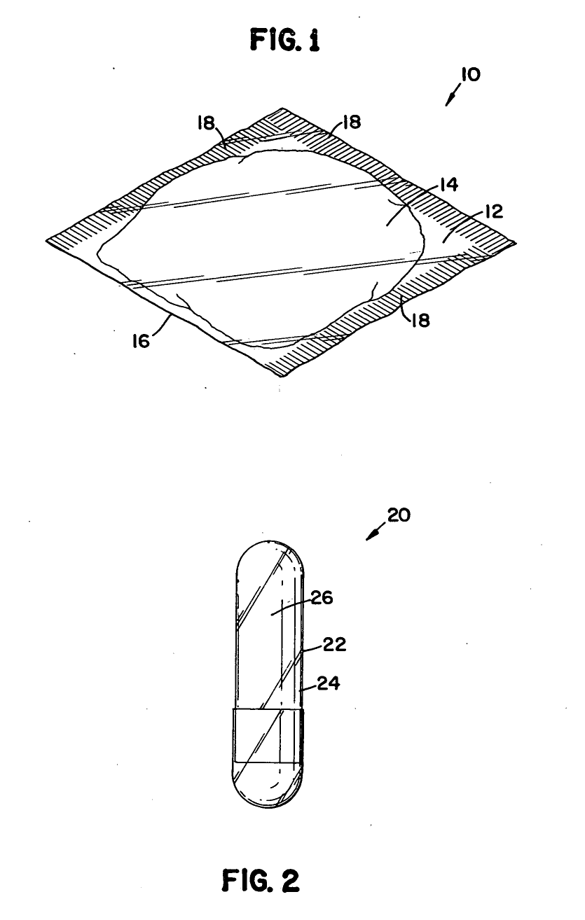

Now referring to FIG. 2, a packaged cleaning composition concentrate is shown at reference number 20. The packaged cleaning composition concentrate 20 is provided as a capsule 22 having a capsule material 24 containing a cleaning composition concentrate 26. The capsule 22 can be introduced into a volume of water, and the capsule material 24 can solubilize, degrade, or disperse to allow contact of the cleaning composition concentrate 26 with the water. The capsule 22 can be advantageous because it may be easier to introduce the capsule 22 through certain geometric configurations such as, the neck of a bottle (e.g., a spray bottle). Accordingly, when one has exhausted or nearly exhausted the contents of a spray bottle containing a cleaning composition, one can fill the spray bottle with water and insert the capsule into the spray bottle, or one can introduce the capsule into the spray bottle and then fill the spray bottle with water. The capsule material 24 can be provided from a water soluble polymer or water dispersible polymer as discussed previously.

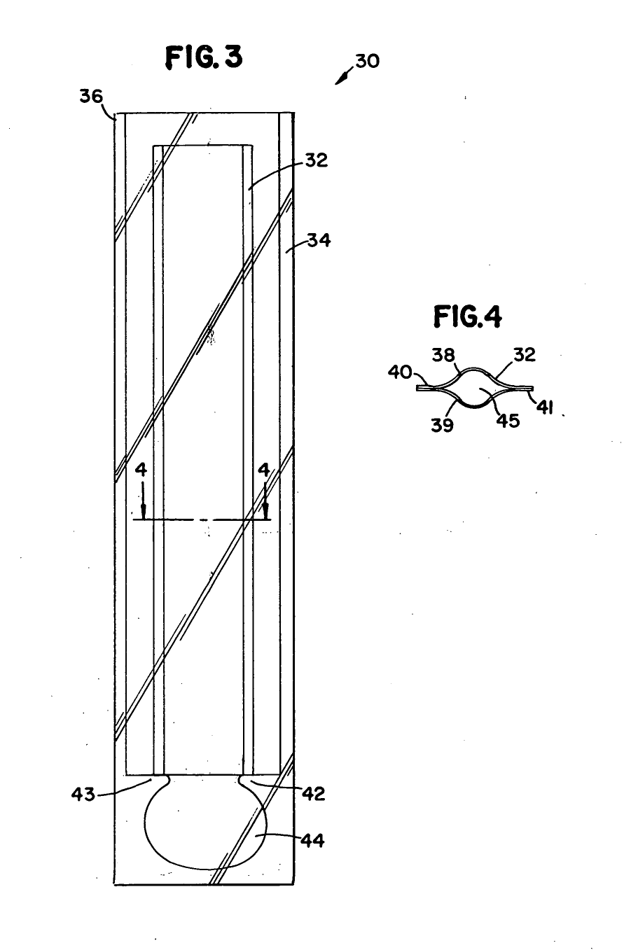

Now referring to FIG. 3, a packaged cleaning composition concentrate is shown at reference number 30. The packaged cleaning composition concentrate 30 includes a substrate 32 containing a cleaning composition concentrate and a film 34 for enclosing and containing the substrate 32 and the concentrate. When it is desirable to remove the substrate 32 from the film 34, one can cut or tear the top portion 36 of the film 34 and remove the substrate 32 therefrom. The substrate 32 can be provided in a form that allows a bottle dip tube to extend therethrough. An exemplary cross section of the substrate 32 is shown in FIG. 4. The substrate 32 can be referred to as a sleeve or as a carrier. The substrate 32 can be provided as a laminate of a first substrate 38 and a second substrate 39. The first substrate 38 and the second substrate 39 can be bonded at the seams 40 and 41. A bottle dip tube can extend through the hollow opening 45. In general, the bottle dip tube refers to the tube extending from a sprayer to the bottom of a bottle, and is used to draw liquid from the bottle to the spray nozzle.

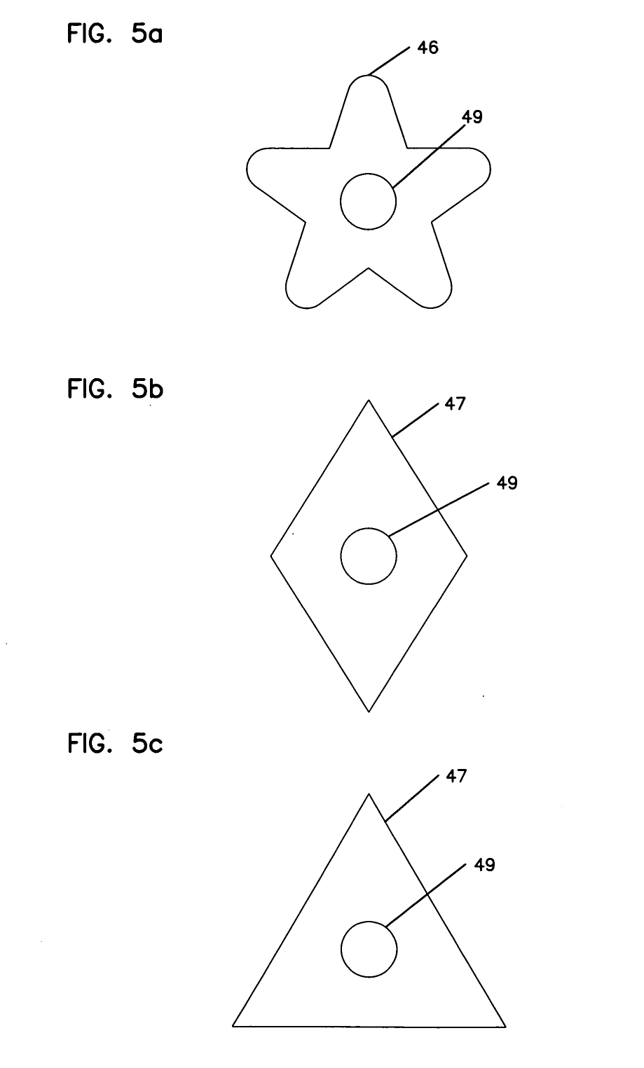

Exemplary sleeves that can be used on dip tubes are shown in FIGS. 5(a)-(c) at reference numbers 46, 47, and 48. Sleeve 46 is shown having a star shape, sleeve 47 is shown having a diamond shape, and sleeve 48 is shown having a triangular shape. The sleeves 46-48 are shown as cross-sectional views and include an opening 49 through which a dip tube can extend. The sleeves 46-48 can be provided as non-wovens. Exemplary disclosures for the manufacture of non-wovens that can be used to form these sleeves or other sleeves having different cross-section shapes include, for example, U.S. Pat. No. 6,576,034, U.S. Pat. No. 5,607,766, U.S. Patent Application Publication No. US2005/0189292, and U.S. Patent Application Publication No. US2005/0153132. The disclosures in these patent publications are incorporated herein by reference. The sleeves 46, 47, and 48 can be characterized as three dimensional non-wovens and can be provided having wicking properties.

Referring to FIG. 3, the film 34 can be provided having shoulders 42 and 43 that prevent the substrate 32 from moving into the open area 44. One can introduce a bottle dip tube through the hollow opening 45 in the substrate 32 so that the tip of the bottle dip tube enters into the opening 44. If the tip of the bottle dip tube has a hook or catch on it, simply pulling the bottle dip tube out of the film 34 through the top portion 36 can cause the substrate 32 to remain on the bottle dip tube thereby providing for separation of the substrate 32 from the film 34. The bottle dip tube containing the substrate 32 can then be introduced into a bottle of water. An exemplary disclosure of a substrate that can be used on a spray bottle dip tube is shown in U.S. Pat. No. 6,250,511 to Kelly, the entire disclosure of which is incorporated herein by reference.

The cleaning composition concentrate can be provided on the substrate 32 as a solid, liquid, or gel. The substrate 32 can be provided in the form of a fabric (e.g., non-woven, woven, or knitted) containing the cleaning composition concentrate as an impregnant or coating. Providing the cleaning composition concentrate as a solid (e.g., powder or aggregate) on the fabric can be advantageous for reducing the transfer of the detergent composition concentrate to other substrates such as the interior of the film 34. The detergent composition concentrate can additionally be provided as a liquid or gel where a sufficient amount of the detergent composition concentrate will remain on the fabric until the fabric is introduced into a body of water such as the inside of a spray bottle.

An advantage of the use of a bottle dip tube for capturing the substrate 32 is the ability for a user to avoid touching the substrate 32 with his or her hands. While it may be advantageous under certain circumstances to avoid touching the substrate 32, the cleaning composition concentrate can be provided as part of a substrate where a user can touch the substrate. That is, in an alternative embodiment, a user can simply remove the substrate from a package or container and introduce the substrate into a volume of water to generate a detergent composition use composition. In addition, the substrate need not be provided in the form of a substrate having a hollow opening. Instead, the substrate can be provided having a single or multiple layer structure. For example, a user can remove the substrate from a package or container that may include multiple substrates, and then place the substrate in a container of water. While it may be desirable under certain circumstances to avoid touching the substrate, the substrate can be constructed so that it can be touched. If the substrate is damp, it may be desirable to avoid touching the substrate to reduce transfer of the concentrate to skin tissue.

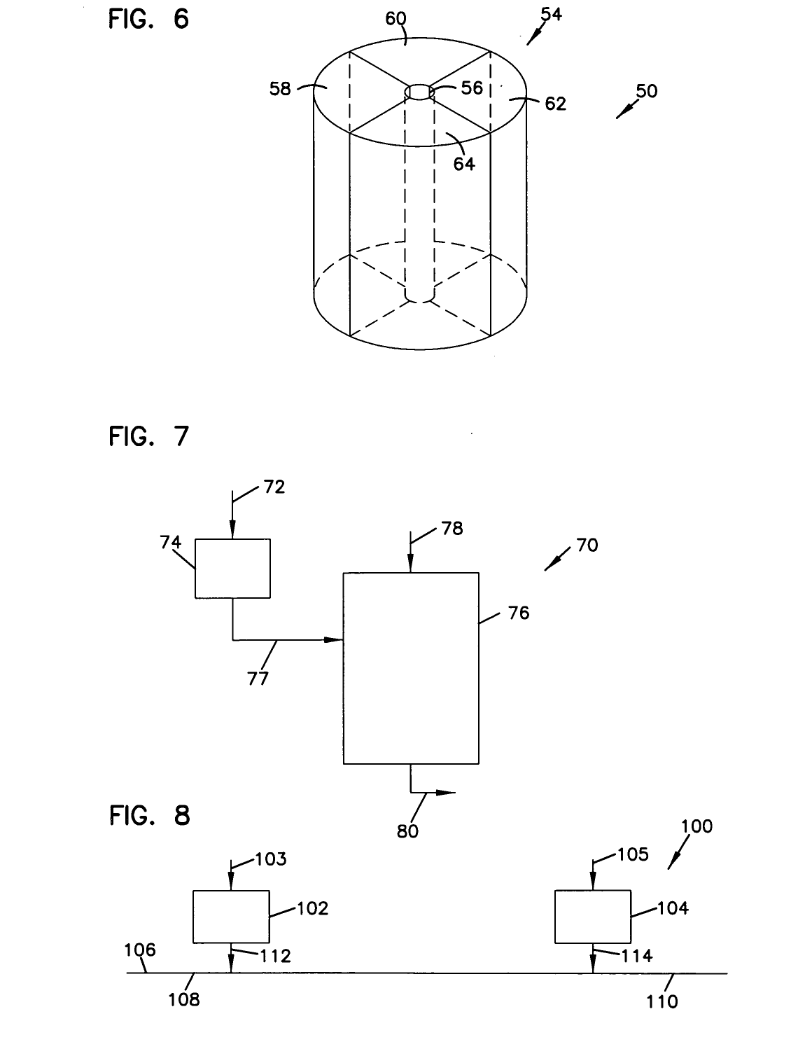

Now referring to FIG. 6, a packaged cleaning composition concentrate is shown at reference number 50. The packaged cleaning composition concentrate 50 is shown as a cartridge 54 having multiple reservoirs containing cleaning composition concentrate. The cartridge 54 can be placed, for example, in the neck of a bottle designed to receive the cartridge 54 and designed to puncture one of the multiple reservoirs at a time. The cartridge 54 is shown having a first reservoir 58, a second reservoir 60, a third reservoir 62, and a fourth reservoir 64. It should be understood that the cartridge can be provided having more or fewer reservoirs. The user of the bottle can insert the cartridge 54 into the neck of the bottle and a bottle dip tube can be provided so that it extends through the opening 56 in the cartridge 54 and a lance accompanying the bottle dip tube can puncture one of the reservoirs thereby allowing the cleaning composition concentrate to flow into the remainder of the bottle which can contain water. Once the cleaning composition is exhausted or used up, the user can rotate the cartridge or rotate the lance and puncture one of the remaining reservoirs in order to allow the cleaning composition concentrate to mix with a new charge of water in the container. An exemplary construction that can use a cartridge is disclosed by U.S. Pat. No. 6,290,100, the entire disclosure of which is incorporated herein by reference.

Now referring to FIG. 7, an exemplary schematic diagram for preparing a cleaning composition use composition from a concentrate is shown at reference number 70. The schematic diagram 70 shows a batch operation. A cleaning composition concentrate 72 can be introduced into a first reservoir 74. For example, a bottle containing the cleaning composition concentrate 72 can be poured into the reservoir 74. The concentrate can then be allowed to flow into a larger reservoir 76 via line 77, and water 78 can be introduced into the larger reservoir 76 to form the use composition. The use composition can be directed, as needed, into a bottle or multiple bottles via the outlet 80. The same bottle used to fill the first reservoir 74 can be repeatedly used to receive the use composition via the outlet 80.

The schematic diagram shown at reference number 70 can be characterized as a batch operation. That is, a quantity of use composition can be prepared from a quantity of concentrate. As the larger reservoir 76 drains after repeated fillings of containers, one can prepare a new batch of use composition from a given quantity of concentrate. A batch operation can be advantageous when the components of the concentrate are not compatible at the concentration provided by the concentrate and have a tendency to phase separate. Although a batch operation can be used when the concentrate has a tendency to phase separate into two or more phases, the batch operation can additionally be used when the concentrate is provided having a single phase.

Now referring to FIG. 8, an exemplary continuous process for forming cleaning composition use composition is shown at reference number 100. To accommodate for the incompatibility of components in the cleaning composition concentrate, a first portion of the cleaning composition concentrate can be provided in the first reservoir 102 via inlet 103 and a second portion of the cleaning composition concentrate can be provided in the second reservoir 104 via inlet 105. Water 106 can be directed through a line 108 to fill a container at the end of the line 110. Pump or aspirators can be used to draw the first concentrate from the first reservoir 102 and the second concentrate from the second reservoir 104 via the lines 112 and 114. If the cleaning composition concentrate can be provided as a single phase, for example if the composition contains sufficient hydrotrope or the components do not phase separate, it may be desirable to provide the exemplary continuous process shown at reference number 100 as having a single reservoir that contains the concentrate. The concentrate can then be pumped or aspirated into the water stream.

It should be appreciated that the concentrate can be provided as a solid, liquid, or gel. In the case of a solid, the cleaning composition concentrate can be provided as a powder, pellet, tablet, granules, or block. In addition, the cleaning composition concentrate can be provided in the various forms as a unit dose. For example, in the context of FIGS. 1-6, the cleaning composition concentrate can be packaged so that the concentrate size is about 0.5 grams to about 50 grams to provide a use composition volume ranging from about 6 ounces to about one gallon. In the context of FIGS. 7 and 8, it may be more advantageous to form larger amounts of the ready to use composition that can be used to fill multiple containers.

### Anionic Surfactant Component

The cleaning composition can contain an anionic surfactant component that includes a detersive amount of an anionic surfactant or a mixture of anionic surfactants. Anionic surfactants are desirable in cleaning compositions because of their wetting and detersive properties. The anionic surfactants that can be used according to the invention include any anionic surfactant available in the cleaning industry. Exemplary groups of anionic surfactants include sulfonates and sulfates. Exemplary surfactants that can be provided in the anionic surfactant component include alkyl aryl sulfonates, secondary alkane sulfonates, alkyl methyl ester sulfonates, alpha olefin sulfonates, alkyl ether sulfates, alkyl sulfates, and alcohol sulfates.

Exemplary alkyl aryl sulfonates that can be used in the cleaning composition can have an alkyl group that contains 6 to 24 carbon atoms and the aryl group can be at least one of benzene, toluene, and xylene. An exemplary alkyl aryl sulfonate includes linear alkyl benzene sulfonate. An exemplary linear alkyl benzene sulfonate includes linear dodecyl benzyl sulfonate that can be provided as an acid that is neutralized to form the sulfonate. Additional exemplary alkyl aryl sulfonates include xylene sulfonate and cumene sulfonate.

Exemplary alkane sulfonates that can be used in the cleaning composition can have an alkane group having 6 to 24 carbon atoms. Exemplary alkane sulfonates that can be used include secondary alkane sulfonates. An exemplary secondary alkane sulfonate includes sodium C₁₄-C₁₇ secondary alkyl sulfonate commercially available as Hostapur SAS from Clariant.

Exemplary alkyl methyl ester sulfonates that can be used in the cleaning composition include those having an alkyl group containing 6 to 24 carbon atoms.

Exemplary alpha olefin sulfonates that can be used in the cleaning composition include those having alpha olefin groups containing 6 to 24 carbon atoms.

Exemplary alkyl ether sulfates that can be used in the cleaning composition include those having between about 1 and about 10 repeating alkoxy groups, between about 1 and about 5 repeating alkoxy groups. In general, the alkoxy group will contain between about 2 and about 4 carbon atoms. An exemplary alkoxy group is ethoxy. An exemplary alkyl ether sulfate is sodium lauric ether ethoxylate sulfate and is available under the name Steol CS-460.

Exemplary alkyl sulfates that can be used in the cleaning composition include those having an alkyl group containing 6 to 24 carbon atoms. Exemplary alkyl sulfates include sodium laurel sulfate and sodium laurel/myristyl sulfate.

Exemplary alcohol sulfates that can be used in the cleaning composition include those having an alcohol group containing about 6 to about 24 carbon atoms.

The anionic surfactant can be neutralized with an alkaline metal salt, an amine, or a mixture thereof. Exemplary alkaline metal salts include sodium, potassium, and magnesium. Exemplary amines include monoethanolamine, triethanolamine, and monoisopropanolamine. If a mixture of salts is used, an exemplary mixture of alkaline metal salt can be sodium and magnesium, and the molar ratio of sodium to magnesium can be between about 3:1 and about 1:1.

The cleaning composition, when provided as a concentrate, can include the anionic surfactant component in an amount sufficient to provide a use composition having desired wetting and detersive properties after dilution with water. In general, the concentrate can be provided as a solid or as a liquid. When the concentrate is provided as a liquid, it can be provided in a form that is readily flowable so that it can be pumped or aspirated. It is additionally desirable to minimize the amount of water while preserving the flowable properties of the concentrate when it is provided as a liquid. The concentrate can contain about 0.1 wt. % to about 30 wt. % of the anionic surfactant component, about 0.5 wt. % to about 25 wt. % of the anionic surfactant component, and about 1 wt. % to about 15 wt. % of the anionic surfactant component.

### Nonionic Surfactant Component

The cleaning composition can contain a nonionic surfactant component that includes a detersive amount of nonionic surfactant or a mixture of nonionic surfactants. Nonionic surfactants can be included in the cleaning composition to enhance grease removal properties. Although the surfactant component can include a nonionic surfactant component, it should be understood that the nonionic surfactant component can be excluded from the detergent composition, if desired. (EO) block copolymers. These surfactants comprise a di-block polymer comprising an EO block and a PO block, a center block of polyoxypropylene units (PO), and having blocks of polyoxyethylene grafted onto the polyoxypropylene unit or a center block of EO with attached PO blocks. Further, this surfactant can have further blocks of either polyoxyethylene or polyoxypropylene in the molecules. An exemplary average molecular weight range of useful surfactants can be about 1,000 to about 40,000 and the weight percent content of ethylene oxide can be about 10-80% by weight.

Additional nonionic surfactants include alcohol alkoxylates. An exemplary alcohol alkoxylate include linear alcohol ethoxylates such as Tomadol™ 1-5 which is a surfactant containing an alkyl group having 11 carbon atoms and 5 moles of ethylene oxide. Additional alcohol alkoxylates include alkylphenol ethoxylates, branched alcohol ethoxylates, secondary alcohol ethoxylates (e.g., Tergitol 15-S-7 from BASF), castor oil ethoxylates, alkylamine ethoxylates, tallow amine ethoxylates, fatty acid ethoxylates, sorbital oleate ethoxylates, end-capped ethoxylates, or mixtures thereof. Additional nonionic surfactants include amides such as fatty alkanolamides, alkyldiethanolamides, coconut diethanolamide, lauramide diethanolamide, cocoamide diethanolamide, polyethylene glycol cocoamide (e.g., PEG-6 cocoamide), oleic diethanolamide, or mixtures thereof. Additional exemplary nonionic surfactants include polyalkoxylated aliphatic base, polyalkoxylated amide, glycol esters, glycerol esters, amine oxides, phosphate esters, alcohol phosphate, fatty triglycerides, fatty triglyceride esters, alkyl ether phosphate, alkyl esters, alkyl phenol ethoxylate phosphate esters, alkyl polysaccharides, block copolymers, alkyl polyglucocides, or mixtures thereof.

When nonionic surfactants are included in the detergent composition concentrate, they can be included in an amount of at least about 0.1 wt. % and can be included in an amount of up to about 15 wt. %. The concentrate can include about 0.5 wt. % to about 12 wt. % or about 2 wt. % to about 10 wt. % of the nonionic surfactant.

### Amphoteric Surfactant Component

Amphoteric surfactants that can be used to provide desired detersive properties. Exemplary amphoteric surfactants that can be used include the betaines, imidazolines, and propinates. Exemplary amphoteric surfactants include sultaines,

### Amphoteric Surfactant Component

Amphoteric surfactants that can be used to provide desired detersive properties. Exemplary amphoteric surfactants that can be used include the betaines, imidazolines, and propinates. Exemplary amphoteric surfactants include sultaines, amphopropionates, amphrodipropionates, aminopropionates, aminodipropionates, amphoacetates, amphodiacetates, and amphohydroxypropylsulfonates.

The detergent composition concentrate can be provided without any amphoteric surfactant. When the detergent composition includes an amphoteric surfactant, the amphoteric surfactant can be included in an amount of about 0.1 wt. % to about 15 wt. %. The concentrate can include about 0.5 wt. % to about 12 wt. % or about 2 wt. % to about 10 wt. % of the amphoteric surfactant.

### Dispersant Component

The detergent composition concentrate can include a dispersant. The dispersant can help provide stability from precipitation at temperatures down to about 40° F., and at temperatures down to freezing.

The dispersant is a component that is conventionally added to cleaning compositions to handle the hardness found in water. Dispersants that can be used according to the invention include those that are referred to as “lime soap dispersants.” In general, it is understood that dispersants have a tendency to interfere with precipitation of anionic surfactants caused by water hardness.

Dispersants that can be used according to the invention can include a polymer and/or an oligomer containing pendant carboxylic acid groups and/or pendant carboxylic acid salt groups. It should be understood that the term “pendant” refers to the groups being present other than in the polymer backbone and/or oligomer backbone. The dispersants can be available as homopolymers or co-polymers or as homoligomers or co-oligomers. Exemplary dispersants include poly(acrylic acid), poly (acrylic acid/maleic acid) co-polymers, poly(maleic acid/olefin) co-polymers, phosphino carboxylated polymers, and mixtures thereof. The dispersants can be soluble or dispersible in the concentrate and can be a component that does not significantly increase the viscosity of the concentrate or of the use solution relative to its absence. The dispersant can be a homopolymer or co-polymer, and can have a molecular weight range of about 300 to about 5,000,000, and can have a molecular weight range of about 2,000 to about 2,000,000, and can have a molecular weight range of about 3,000, to about 500,000. The dispersant can include repeating units based upon acrylic acid, maleic acid, polyols, olefins, and mixtures thereof. An exemplary dispersant is a maleic anhydride/olefin co-polymer. An exemplary maleic anhydride/olefin co-polymer is available from Rohm & Haas under the name of Acusol 460N. An exemplary polyacrylic acid sodium salt having a molecular weight of about 4,500 is available from Rohm & Haas under the name Acusol 434N. An exemplary acrylic acid/maleic acid co-polymer having a molecular weight of about 3,200 is available from Rohm & Haas under the Acusol 448. An exemplary acrylic acid/maleic acid sodium salt having a molecular weight of about 70,000 is available from Rohm & Haas under the name Acusol 479N. An exemplary acrylic acid/maleic acid sodium salt having a molecular weight of about 40,000 is available from Rohm & Haas under the name Acusol 505N. In general, if the dispersant is provided as an acid, its pH may be adjusted to neutral or alkaline. The pH adjustment may be provided prior to forming the concentrate or during the formation of the concentrate. In addition, the pH adjustment may occur at any time prior to or during dilution with the water of dilution to provide the use solution.

The dispersant can be provided in the detergent composition concentrate in an amount sufficient, when taken in consideration of the amount of sheeting agent and/or humectant, to provide resistance to precipitation of the anionic surfactant component when diluted with hard water. In general, the concentrate can contain about 0.01 wt. % to about 10 wt. % dispersant, about 0.1 wt. % to about 5 wt. % dispersant, and about 0.2 wt. % to about 4 wt. % dispersant.

### Sheeting Agent and Humectant Component

The detergent composition concentrate can include a sheeting agent, a humectant, or a combination of sheeting agent and humectant. It is believed that the combination of the dispersant and at least one of a sheeting agent or a humectant can provide water hardness anti-precipitant properties. The combination of the dispersant and at least one of a sheeting agent or a humectant can provide the use composition with resistance to precipitation of the anionic surfactant component caused by hardness in the water. Exemplary water hardness anti-precipitant mixtures disclosed in U.S. Patent Application Publication No. US2004/0154640 that was filed with the United States Patent and Trademark Office on Nov. 25, 2003. The entire disclosure of U.S. Patent Application Publication No. US2004/0154640 is incorporated herein by references.

The sheeting agent or humectant can be any component that provides a desired level of sheeting action and, when combined with the dispersant, creates a resistance to precipitation of the anionic surfactant component in the presence of hard water.

Exemplary sheeting agents that can be used according to the invention include surfactant including nonionic block copolymers, alcohol alkoxylates, alkyl polyglycosides, zwitterionics, anionics, and mixtures thereof. Additional exemplary sheeting agents include alcohol ethoxylates; alcohol propoxylates; alkylphenol ethoxylate-propoxylates; alkoxylated derivatives of carboxylic acids, amines, amides and esters; and ethylene oxide-propylene oxide copolymers. Exemplary ethylene oxide-propylene oxide polymers include those available under the name Pluronic, Pluronic R, Tetronic, and Tetronic R from BASF.

Exemplary nonionic block copolymer surfactants include polyoxyethylene-polyoxypropylene block copolymers. Exemplary polyoxyethylene-polyoxypropylene block copolymers that can be used have the formulae:
(EO)ₓ(PO)_{y}(EO)ₓ
(PO)_{y}(EO)ₓ(PO)_{y}
(PO)_{y}(EO)ₓ(PO)_{y}(EO)ₓ(PO)_{y}
wherein EO represents an ethylene oxide group, PO represents a propylene oxide group, and x and y reflect the average molecular proportion of each alkylene oxide monomer in the overall block copolymer composition. Preferably, x is from about 10 to about 130, y is about 15 to about 70, and x plus y is about 25 to about 200. It should be understood that each x and y in a molecule can be different. The total polyoxyethylene component of the block copolymer is preferably at least about 20 mol-% of the block copolymer and more preferably at least about 30 mol-% of the block copolymer. The material preferably has a molecular weight greater than about 1,500 and more preferably greater than about 2,000. Although the exemplary polyoxyethylene-polyoxypropylene block copolymer structures provided above have 3 blocks and 5 blocks, it should be appreciated that the nonionic block copolymer surfactants according to the invention can include more or less than 3 and 5 blocks. In addition, the nonionic block copolymer surfactants can include additional repeating units such as butylene oxide repeating units. Furthermore, the nonionic block copolymer surfactants that can be used according to the invention can be characterized heteric polyoxyethylene-polyoxypropylene block copolymers. Exemplary sheeting agents that can be used according to the invention are available from BASF under the name Pluronic, and an exemplary EO-PO co-polymer that can be used according to the invention is available under the name Pluronic N3.

A desirable characteristic of the nonionic block copolymers is the cloud point of the material. The cloud point of nonionic surfactant of this class is defined as the temperature at which a 1 wt-% aqueous solution of the surfactant turns cloudy when it is heated. BASF, a major producer of nonionic block copolymers in the United States recommends that rinse agents be formulated from nonionic EO-PO sheeting agents having both a low molecular weight (less than about 5,000) and having a cloud point of a 1 wt-% aqueous solution less than the typical temperature of the aqueous rinse. It is believed that one skilled in the art would understand that a nonionic surfactant with a high cloud point or high molecular weight would either produce unacceptable foaming levels or fail to provide adequate sheeting capacity in a rinse aid composition.

The alcohol alkoxylate surfactants that can be used as sheeting agents according to the invention can have the formula:
R(AO)ₓ-X
wherein R is an alkyl group containing 6 to 24 carbon atoms, AO is an alkylene oxide group containing 2 to 12 carbon atoms, x is 1 to 75, and X is hydrogen or an alkyl or aryl group containing 1-12 carbon atoms. An exemplary alcohol alkoxylate that can be used is available under the name Plurafac LF 303 from BASF. The alkylene oxide group can be ethylene oxide, propylene oxide, butylene oxide, or mixture thereof. In addition, the alkylene oxide group can include a decylene oxide group as a cap. In addition, the alcohol alkoxylate can be characterized as having an x value of 1 to 20.

The alkyl polyglycoside surfactants that can be used as sheeting agents according to the invention can have the formula:
(G)ₓ-O-R
wherein G is a moiety derived from reducing saccharide containing 5 or 6 carbon atoms, e.g., pentose or hexose, R is a fatty aliphatic group containing 6 to 24 carbon atoms, and x is the degree of polymerization (DP) of the polyglycoside representing the number of monosaccharide repeating units in the polyglycoside. The value of x can be between about 0.5 and about 10. R can contain 10-16 carbon atoms and x can be 0.5 to 3.

The zwitterionic surfactants that can be used as sheeting agents according to the invention include β-N-alkylaminopropionates, N-alkyl-β-iminodipropionates, imidazoline carboxylates, N-alkylbetaines, sulfobetaines, sultaines, amine oxides and polybetaine polysiloxanes. Preferred polybetaine polysiloxanes have the formula: 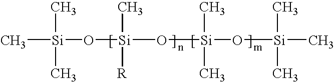
wherein R is 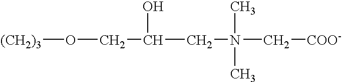
n is 1to 100 and m is 0 to 100, preferably 1 to 100. Preferred polybetaine polysiloxanes are available under the name ABIL® from Goldschmidt Chemical Corp. Preferred amine oxides that can be used include alkyl dimethyl amine oxides containing alkyl groups containing 6 to 24 carbon atoms. A preferred amine oxide is lauryl dimethylamine oxide.

The anionic surfactants that can be used as sheeting agents according to the invention include carboxylic acid salts, sulfonic acid salts, sulfuric acid ester salts, phosphoric and polyphosphoric acid esters, perfluorinated anionics, and mixtures thereof. Exemplary carboxylic acid salts include sodium and potassium salts of straight chain fatty acids, sodium and potassium salts of coconut oil fatty acids, sodium and potassium salts of tall oil acids, amine salts, sarcosides, and acylated polypeptides. Exemplary sulfonic acid salts include linear alkylbenzenesulfonates, C₁₃-C₁₅ alkylbenzenesulfonates, benzene cumenesulfonates, toluene cumenesulfonates, xylene cumenesulfonates, ligninsulfonates, petroleum sulfonates, N-acyl-n-alkyltaurates, paraffin sulfonates, secondary n-alkanesulfonates, alpha-olefin sulfonates, sulfosuccinate esters, alkylnaphthalenesulfonates, and isethionates. Exemplary sulfuric acid ester salts include sulfated linear primary alcohols, sulfated polyoxyethylenated straight-chain alcohols, and sulfated triglyceride oils.

Exemplary surfactants which can be used as sheeting agents according to the invention are disclosed in Rosen, Surfactants and Interfacial Phenomena, second edition, John Wiley & sons, 1989, the entire document being incorporated herein by reference. Humectants that can be used according to the invention include those substances that exhibit an affinity for water and help enhance the absorption of water onto a substrate. If the humectant is used in the absence of a sheeting agent, the humectant should be capable of cooperating with the dispersant to resist precipitation of the anionic surfactant in the presence of hard water. Exemplary humectants that can be used according to the invention include glycerine, propylene glycol, sorbitol, alkyl polyglycosides, polybetaine polysiloxanes, and mixtures thereof. The alkyl polyglycosides and polybetaine polysiloxanes that can be used as humectants include those described previously as sheeting agents.

When the humectant is incorporated into the cleaning composition, it can be used in an amount based upon the amount of sheeting agent used. In general, the weight ratio of humectant to sheeting agent can be greater than 1:3, and can be provided at between about 5:1 and about 1:3. It should be appreciated that the characterization of the weight ratio of humectant to sheeting agent indicates that the lowest amount of humectant to sheeting agent is 1:3, and that more humectant relative to the same amount of sheeting agent can be used. The weight ratio of humectant to sheeting agent can be between about 4:1 and about 1:2, and can be between about 3:1 and about 1:1. When using a humectant in the cleaning composition, it is preferable that the sheeting agent and the humectant are not the same chemical molecule. Although alkyl polyglycosides and polybetaine polysiloxanes are identified as both sheeting agents and humectants, it should be understood that the cleaning composition preferably does not have a particular alkyl polyglycoside functioning as both the sheeting agent and the humectant, and preferably does not have a specific polybetaine polysiloxane functioning as the sheeting agent and the humectant. It should be understood, however, that different alkyl polyglycosides and/or different polybetaine polysiloxanes can be used as sheeting agents and humectants in a particular cleaning composition.

It is understood that certain components that are characterized as humectants have been used in prior compositions as, for example, processing aids, hydrotropes, solvents, and auxiliary components. In those circumstances, it is believed that the component has not been used in an amount or an in environment that provides for reducing water solids filming in the presence of high solids containing water. The use of humectants in a rinse agent composition is described in U.S. Pat. No. 6,673,760, the entire disclosure of which is incorporated herein by reference.

The concentrate can include an amount of sheeting agent and/or humectant that cooperates with the dispersant to resist precipitation of the anionic surfactant by hard water. The concentrate can contain about 0.001 wt. % to about 10 wt. % of the sheeting agent and/or humectant, about 0.01 wt. % to about 8 wt. % of the sheeting agent and/or humectant, and about 0.05 wt. % to about 5 wt. % of the sheeting agent and/or humectant.

The amounts of dispersant and at least one of sheeting agent or humectant provided in the cleaning composition can be controlled to handle the water hardness levels expected from various localities as a result of the dilution of the concentrate to a use solution. In general, it is expected that the weight ratio of the dispersant to the total sheeting agent and/or humectant can be about 1:75 to about 75:1, about 1:30 to about 30:1, about 1:25 to about 25:1, about 1:15 to about 15:1; about 1:10 to about 10:1, and about 1:5 to about 5:1.

### Water Component

The concentrate can be provided in the form of a solid, a liquid, or gel, or a combination thereof. The concentrate can be formulated without any water or can be provided with a relatively small amount of water in order to reduce the expense of transporting the concentrate. When the concentrate is provided as a liquid, it may be desirable to provide it in a flowable form so that it can be pumped or aspirated. It has been found that it is generally difficult to accurately pump a small amount of a liquid. It is generally more effective to pump a larger amount of a liquid. Accordingly, although it is desirable to provide the concentrate with as little as possible in order to reduce transportation costs, it is also desirable to provide a concentrate that can be dispensed accurately. As a result, a concentrate according to the invention, when it includes water, it can include water in an amount of about 0.1 wt. % to about 99 wt. %, about 30 wt. % to about 95 wt. %, and about 40 wt. % to about 90 wt. %.

It should be understood that the water provided as part of the concentrate can be relatively free of hardness. It is expected that the water can be deionized to remove a portion of the dissolved solids. The concentrate is then diluted with water available at the locale or site of dilution and that water may contain varying levels of hardness depending upon the locale. Although deionized is preferred for formulating the concentrate, the concentrate can be formulated with water that has not been deionized. That is, the concentrate can be formulated with water that includes dissolved solids, and can be formulated with water that can be characterized as hard water.

Service water available from various municipalities has varying levels of hardness. It is generally understood that the calcium, magnesium, iron, manganese, or other polyvalent metal cations that may be present can cause precipitation of the anionic surfactant. In general, because of the expected large level of dilution of the concentrate to provide a use solution, it is expected that service water from certain municipalities can have a greater impact on the potential for anionic surfactant precipitation than the water from other municipalities. As a result, it can be desirable to provide a concentrate that can handle the hardness levels found in the service water of various municipalities.

When the hardness level is considered to be fairly high, it can be difficult to handle the hardness using traditional builders because of the large amount of water of dilution used to dilute the concentrate to form the use solution. Because builders have a tendency to act in a molar relationship with cationic salts, it is expected that the concentrate would require a large amount of a builder component if the builder component was the only component responsible for handling the hardness. Accordingly, even if it is possible to incorporate an amount of builder into the concentrate to prevent precipitation of the anionic surfactant component, it can be desirable to provide a concentrate that does not require so much builder to handle the hardness levels found in the service water of various municipalities.

The water of dilution that can be used to dilute the concentrate can be characterized as hard water when it includes at least 1 grain hardness. It is expected that the water of dilution can include at least 5 grains hardness, at least 10 grains hardness, or at least 20 grains hardness.

The concentrate can be diluted with the water of dilution in order to provide a use solution having a desired level of detersive properties. If the concentrate contains a large amount of water, the concentrate can be diluted with the water of dilution at a weight ratio concentrate to water of dilution of at least 1:1 to provide a desired use solution. If the concentrate includes no water or very little water, the concentrate can be diluted at a weight ratio of concentrate to water of dilution of up to about 1:1000 in order to provide a desired use composition. The weight ratio of concentrate to water of dilution can be about 1:5 to about 1:500, about 1:10 to about 1:400, and about 1:20 to about 1:300.

The concentrate can be characterized based on the weight percent actives. The actives refers to the non-aqueous part of the composition. It can be desirable to provide the concentrate with a fairly high weight percent actives so that the concentrate can be diluted at the above-identified dilution ratios to provide a desired use composition. The concentrate can contain at least about 1 wt. % actives, and preferably at least about 5 wt. % actives, and more preferably at least about 10 wt. % actives. The concentrate can additionally contain at least about 20 wt. % active, at least about 40 wt. % actives, or at least about 50 wt. % actives. If the concentrate is provided without any water, it can be characterized as 100% actives. The concentrate can contain less than 99 wt. % actives, less than 90 wt. % actives, or less than about 80 wt. % actives.

### Other Components

The detergent composition can include an organic solvent to modify cleaning properties and/or modify the evaporation rate of water from the surface that is cleaned. In general, the properties of modifying cleaning and modifying evaporation can be balanced depending upon the application of the use solution. In addition, the cleaning composition can include a single organic solvent or a mixture of organic solvents.

Exemplary organic solvents that can be used include hydrocarbon or halogenated hydrocarbon moieties of the alkyl or cycloalkyl type, and have a boiling point well above room temperature, i.e., above about 20° C.

Considerations for selecting organic solvents include cleaning properties and aesthetic considerations. For example, kerosene hydrocarbons function quite well for grease cutting in the present compositions, but can be malodorous. Kerosene must be exceptionally clean before it can be used, even in commercial situations. For home use, where malodors would not be tolerated, the formulator would be more likely to select solvents which have a relatively pleasant odor, or odors which can be reasonably modified by perfuming.

The C₆-C₉ alkyl aromatic solvents, especially the C₆-C₉ alkyl benzenes, preferably octyl benzene, exhibit excellent grease removal properties and have a low, pleasant odor. Likewise the olefin solvents having a boiling point of at least about 100° C., especially alpha-olefins, preferably 1-decene or 1-dodecene, are excellent grease removal solvents.

Generically, the glycol ethers useful herein have the formula R¹O—(R²O—)ₘ1H wherein each R¹ is an alkyl group which contains from about 1 to about 8 carbon atoms, each R² is either ethylene or propylene, and m is a number from 1 to about 3. Exemplary glycol ethers include monopropyleneglycolmonopropyl ether, dipropyleneglycolmonobutyl ether, monopropyleneglycolmonobutyl ether, ethyleneglycolmonohexyl ether, ethyleneglycolmonobutyl ether, diethyleneglycolmonohexyl ether, monoethyleneglycolmonohexyl ether, monoethyleneglycolmonobutyl ether, and mixtures thereof.

Solvents such as pine oil, orange terpene, benzyl alcohol, n-hexanol, phthalic acid esters of C₁₋₄ alcohols, butoxy propanol, Butyl Carbitol® and 1(2-n-butoxy-1-methylethoxy)propane-2-ol (also called butoxy propoxy propanol or dipropylene glycol monobutyl ether), hexyl diglycol (Hexyl Carbitol®), butyl triglycol, diols such as 2,2,4-trimethyl-1,3-pentanediol, and mixtures thereof, can be used.

The concentrate can include the organic solvent component in an amount to provide the desired cleaning and evaporative properties. In general, the amount of solvent should be limited so that the use solution is in compliance with volatile organic compound (VOC) regulations for a particular class of cleaner. In addition, it should be understood that the organic solvent is an optional component and need not be incorporated into the concentrate or the use solution according to the invention. When the organic solvent is included in the concentrate, it can be provided in an amount of about 0.1 wt. % to about 99 wt. %, about 5 wt. % to about 70 wt. %, and about 10 wt. % to about 60 wt. %, and about 30 wt. % to about 50 wt. %.

It can be desirable to provide the use solution with a relatively neutral or alkaline pH. In many situations, it is believed that the presence of hard water as water of dilution will cause the use solution to exhibit a neutral or alkaline pH. In order to ensure a relatively neutral or alkaline pH, a buffer can be incorporated into the concentrate. In general, the amount of buffer should be sufficient to provide the use composition with a pH in the range of about 6 to 14, about 7 to 12, or about 9 to 11.

The buffer can include an alkalinity source. Exemplary alkaline buffering agents include alkanolamines. An exemplary alkanolamine is beta-aminoalkanol and 2-amino-2-methyl-1-propanol (AMP).

Preferred alkanolamines are beta-aminoalkanol compounds. They serve primarily as solvents when the pH is about 8.5, and especially above about 9.0. They also can provide alkaline buffering capacity during use. Exemplary beta-aminoalkanols are 2-amino-1-butanol; 2-amino-2-methyl-1-propanol; and mixtures thereof. The most preferred beta-aminoalkanol is 2-amino-2-methyl-1-propanol since it has the lowest molecular weight of any beta-aminoalkanol which has the amine group attached to a tertiary carbon atom. The beta-aminoalkanols preferably have boiling points below about 175° C. Preferably, the boiling point is within about 5° C. of 165° C.

Beta-aminoalkanols, and especially monoethanolamine and the preferred 2-amino-2-methyl-1-propanol, are surprisingly volatile from cleaned surfaces considering their relatively high molecular weights. It is found that levels below an equivalent of about 0.010% 2-amino-2-methyl-1-propanol are insufficient to provide the necessary buffering capacity necessary to maintain the pH of the formulations within a narrow range.

Other suitable alkalinity agents that can also be used include alkali metal hydroxides, e.g., sodium, potassium, etc., and carbonates or sodium bicarbonates, and silicates, e.g., potassium silicates. An exemplary potassium hydroxide is available as flake (90%) or bead. An exemplary potassium silicate is available under the name Kasil #6 (39.15%). Water-soluble alkali metal carbonate and/or bicarbonate salts, such as sodium bicarbonate, potassium bicarbonate, potassium carbonate, cesium carbonate, sodium carbonate, and mixtures thereof, can be added to the composition of the present invention in order to improve the filming/streaking when the product is wiped dry on the surface, as is typically done in glass cleaning. Preferred salts are sodium carbonate, potassium carbonate, sodium bicarbonate, potassium bicarbonate, their respective hydrates, and mixtures thereof.

Contrary to the teachings of U.S. Pat. No. 6,420,326, the concentrate can include a buffering capacity greater than the equivalent of 0.050 wt. % 2-amino-2-methyl-1-propanol without experiencing deleterious streaking as a glass cleaner composition. In addition, the concentrate can include a buffering capacity greater than the equivalent of 0.070 wt. % of 2-amino-2-methyl-1-propanol, and greater than the equivalent of 0.1 wt. % of 2-amino-2-methyl-1-propanol.

The cleaning composition according to the invention can include complexing or chelating agents that aid in reducing the harmful effects of hardness components in service water. Typically, calcium, magnesium, iron, manganese, or other polyvalent metal cations, present in service water, can interfere with the action of cleaning compositions. A chelating agent can be provided for complexing with the metal cation and preventing the complexed metal cation from interfering with the action of an active component of the rinse agent. Both organic and inorganic chelating agents are common. Inorganic chelating agents include such compounds as sodium pyrophosphate, sodium tripolyphosphate, and tripotassium pyrophosphate. Organic chelating agents include both polymeric and small molecule chelating agents. Polymeric chelating agents commonly comprise ionomer compositions such as polyacrylic acids compounds. Small molecule organic chelating agents include amino-carboxylates such as salts of ethylenediaminetetracetic acid (EDTA) and hydroxyethylenediaminetetracetic acid, nitrilotriacetic acid, ethylenediaminetetrapropionates, triethylenetetraminehexacetates, and the respective alkali metal ammonium and substituted ammonium salts thereof, citrates such as sodium citrate, and trisodium methyl glycine diacetic acid (MGDA). MGDA is available under the name Trilon M from BASF. Phosphonates are also suitable for use as chelating agents in the composition of the invention and include ethylenediamine tetra(methylenephosphonate), nitrilotrismethylenephosphonate, diethylenetriaminepenta(methylene phosphonate), hydroxyethylidene diphosphonate, and 2-phosphonobutane-1, 2, 4-tricarboxylic acid. Preferred chelating agents include the phosphonates amino-carboxylates. These phosphonates commonly contain alkyl or alkylene groups with less than 8 carbon atoms.

It should be understood that the concentrate can be provided without a component conventionally characterized as a builder, a chelating agent, or a sequestrant. Nevertheless, these components can advantageously be incorporated into the cleaning composition. When these components are included, they can be provided in an amount less than necessary or sufficient to handle the hardness in the water resulting from the water of dilution mixing with the concentrate to form the use solution when the water of dilution is considered to be fairly hard water and the ratio of water of dilution to the concentrate is fairly high.

The detergent composition concentrate can include a hydrotrope. In general, the hydrotrope can be present to help keep the components of the composition together. Exemplary hydrotropes include the sodium, potassium ammonium and sodium, potassium ammonium salts of the alkyl sarcosinates, sodium xylene sulfonate, and sodium cumene sulfonate.

Although hydrotropes can be useful to help hold components of a composition together in a single phase, it should be understood that the hydrotrope is an optional component and need not be included in the detergent composition concentrate. Furthermore, the detergent composition concentrate can be provided having multiple phases. For example, when preparing a batch of the use composition, it may be acceptable in a particular application for the detergent composition concentrate to be available in multiple phases as long as the use composition is provided having a single phase. When the detergent composition concentrate includes a hydrotrope, the hydrotrope can be provided in a amount of about 0.1 wt. % to about 10 wt. %, about 0.5 wt. % to about 5 wt. %, or about 1 wt. % to about 3 wt. %.

The detergent composition concentrate can include a corrosion inhibitor to help protect metals that may contact the use composition from corrosion. Exemplary types of corrosion inhibitors include those corrosion inhibitors that protect aluminum, copper, steel, brass, and iron. Exemplary corrosion inhibitors include amine borate, neutralized salt of alkyl amido carboxylic acid and alkanolamine, neutralized salt of alkyl amido carboxylic acid and triethanolamine, neutralized salt of octane phosphonic acid and alkanolamine, potassium silicate, sodium silicate, sodium metasilicate, phosphate ester, alkyl aryl sulfonate, calcium sulfonate, cocoamide diethylamine, and mixtures thereof. Exemplary corrosion inhibitors are available under the names Mazon RI 325 from BASF; Hostacor 2732, Hostacor IS, Hostacor IT, and Hostaphat OPS 100 from Clariant; Berol 525 and Berol 725 from Akzol Nobel; Klearfac AA270 and Maphos from BASF; Rodafac from Rhodia; Cobratec 948 and Cobratec AL250 from PMC; and alkylaryl sulfonate calcium sulfonate from Pilot. The detergent composition concentrate can be provided without any corrosion inhibitor. If the detergent composition concentrate includes a corrosion inhibitor, it is preferably included in an amount sufficient to provide corrosion inhibition properties. The detergent composition concentrate can include the corrosion inhibitor in an amount of about 0.05 wt. % to about 30 wt. %, about 0.02 wt. % to about 20 wt. %, and about 0.5 wt. % to about 10 wt. %.

Optional ingredients which can be included in the cleaning composition of the invention in conventional levels for use include processing aids, dyes, pigments fillers, optical brighteners, germicides, bleaches, bleach activators, fragrances, viscosity modifiers, preservatives, and UV protectants.

The ready to use composition and/or the use solution can be foamed during application onto a surface. In the case of a glass cleaner, a foam is generally desirable to provide the composition additional hang time. That is, it is generally desirable to allow the cleaning composition to remain in place on a surface that may be vertical until a user has the opportunity to wipe the cleaner on the surface to provide cleaning. It is believed the cleaning composition can be foamed without the need for certain types of foaming agents such as thickeners. In fact, it is believed that certain thickeners may have an adverse affect on cleaning when used to clean a glass surface if the thickener has a tendency to cause smearing, streaking, or leave a film on the glass surface. Accordingly, thickeners can be excluded from the composition according to the invention. Specific types of thickeners that can be excluded include those thickeners that provide a thickening effect by increasing the viscosity by at least 50 cP. When used as a window cleaner, the cleaning composition can be wiped away, without a water rinse, to provide a streak free glass surface.

An exemplary detergent composition concentrate can be formulated accordingly to Table 1.

TABLE 1

| | First Range | Second Range | Third Range |
|---|---|---|---|
| Component | (wt. %) | (wt. %) | (wt. %) |
| water | 0.1-99 | 30-95 | 40-90 |
| anionic surfactant | 0.1-30 | 0.5-25 | 1-15 |
| nonionic surfactant | 0.1-15 | 0.5-12 | 2-10 |
| amphotertic surfactant | 0.1-15 | 0.5-12 | 2-10 |
| dispersant | 0.01-10 | 0.1-5 | 0.2-4 |
| sheeting agent | 0.001-10 | 0.01-8 | 0.05-5 |
| humectant | 0.001-10 | 0.01-8 | 0.05-5 |
| organic solvent | 0.1-99 | 5-70 | 10-60 |
| hydrotrope | 0.001-10 | 0.5-5 | 1-3 |
| corrosion inhibitor | 0.05-10 | 0.2-20 | 0.5-10 |

The cleaning composition can be prepared at a first location and shipped or transported to a second location for dilution. The second location can be provided with a water source that includes hardness. An exemplary type of second location is a commercial store where the concentrate is diluted, packaged, and distributed to customers. The second location can be another facility that provides for further dilution and distribution of the product. In addition, the second location can be a job site, such as, a restaurant, grocery store, hotel or other building requiring janitorial services. In addition, it should be understood that there can be multiple locations where dilution occurs. For example, an intermediary dilution can occur at the second location, and the final dilution to a use solution can be provided by the consumer at about the time the detergent composition is used for cleaning.

The detergent composition concentrate can be prepared by mixing the components together. When an organic solvent is desired in the detergent composition concentrate, the components of the detergent composition concentrate, other than the organic solvent, can be combined together by mixing, and then the organic solvent can be added separately. In certain formulations, it is possible that the detergent composition concentrate containing the organic solvent may have a tendency to phase separate. A hydrotrope can be used to help reduce phase separation.

The detergent composition, when provided as a use solution, can be applied to a surface or substrate for cleaning in a variety of forms. Exemplary forms include as a spray and as a foam. In the case of a glass cleaner, it may be desirable to provide the use solution as a foam in order to hinder running of the use solution down a vertical window. It is believed that a pump foamer can be used to create a foam for application to a surface or substrate without the need for propellants or other blowing agents. The foam can be characterized as a mechanically generated foam rather than a chemically generated foam when a hand or finger pump is used to create the foam. An exemplary foaming head that can be used with the detergent composition can be obtained from Zeller in Germany.

It is believed that that cleaning composition can be used as a glass cleaner for cleaning glass surfaces including windows and mirrors. In addition, it is believed that the cleaning composition can be used as a hard surface cleaner, a bathroom cleaner, a dishwash detergent, a floor cleaner, a countertop cleaner, and a metal cleaner. In addition, it is believed that the detergent composition can be used in a car wash facility for cleaning glass, for washing the car, for prewash applications, and for metal brightening. It should be understood that the cleaning composition can be applied directly to a surface such as a glass surface and wiped away to provide a streak free surface. In addition, the detergent composition can be rinsed from a surface with water.

Several exemplary concentrate compositions are provided in the following tables. It should be understood that the organic solvent can be provided separate from the remaining components in the exemplary compositions until it is desired to combine the organic solvent with the remaining components.

TABLE 2

| Components | Wt % |
|---|---|
| organic solvent¹ | 39 |
| buffer² | 3 |
| dispersant³ | 7.9 |
| sheeting agent⁴ | .79 |
| dye⁵ | .24 |
| nonionic surfactant⁶ | 6 |
| anionic surfactant⁷ | 34 |
| chelant⁸ | 7.9 |
| fragrance⁹ | 1 |

¹propylene glycol N-propyl ether (PNP)
²2-amino 2-methyl 1-propanol, 95% liq.
³Rohm & Haas Acusol 460N, 25%
⁴BASF Plurafac LF 303
⁵Acid Blue 62 + 80
⁶Tomah Tomadol 1-5 (Linear Alcohol Ethoxylate)
⁷Sodium Lauryl Sulfate LCP, 30%
⁸Na3MGDA, BASF Trilon M, 40% Soln.
⁹SZ 15162, Floral Lavender

TABLE 3

| Components | Wt % |
|---|---|
| organic solvent | 36 |
| buffer | .2 |
| dispersant | 7.3 |
| sheeting agent | .7 |
| dye | .2 |
| nonionic surfactant | 5.5 |
| anionic surfactant | 31.6 |
| alkalinity source¹⁰ | .7 |
| corrosion inhibitor¹¹ | 1.8 |
| chelant | 10.9 |
| hydrotrope¹² | 4.3 |
| fragrance | .9 |

¹⁰potassium hydroxide, flake, 90%
¹¹Potassium Silicate (Kasil #6), 39.15%
¹²Sodium Xylene sulfonate 40%

TABLE 4

| Components | Wt % |
|---|---|
| organic solvent | 36 |
| buffer | 2.8 |
| dispersant | 10.1 |
| sheeting agent | 1 |
| dye | .2 |
| nonionic surfactant | 5.6 |
| anionic surfactant | 31.8 |
| chelant | 10.9 |
| corrosion inhibitor¹³ | .6 |
| fragrance | .9 |

¹³Sodium Metasilicate, Pentahydrate

TABLE 5

| Components | Wt % |
|---|---|
| organic solvent | 36 |
| buffer | .2 |
| dispersant | 7.9 |
| sheeting agent | .9 |
| dye | .2 |
| nonionic surfactant | 5.5 |
| anionic surfactant | 31.3 |
| alkalinity source | .7 |
| corrosion inhibitor | 1.8 |
| Chelant | 10.8 |
| hydrotrope | 4.3 |
| fragrance | .9 |

TABLE 6

| Components | Wt % |
|---|---|
| organic solvent | 36 |
| buffer | .2 |
| dispersant | 7.2 |
| sheeting agent | 1 |
| dye | .2 |
| nonionic surfactant | 5.5 |
| anionic surfactant | 31.4 |
| alkalinity source | .7 |
| corrosion inhibitor | 1.8 |
| Chelant | 10.8 |
| hydrotrope | 4.5 |
| fragrance | .9 |

TABLE 7

| Components | Wt % |
|---|---|
| organic solvent | 27.2 |
| anionic surfactant | 23.9 |
| buffer | 2.0 |
| sheeting agent | 0.7 |
| nonionic surfactant¹⁴ | 4.2 |
| corrosion inhibitor¹⁵ | 16.1 |
| chelant | 14.5 |
| dispersant | 5.5 |
| fragrance | 0.7 |
| hydrotrope | 4.8 |
| dye | 0.2 |

¹⁴Tergitol 15-S-7 from Dow
¹⁵Mazon RI 325 from BASF

The above specification, examples and data provide a complete description of the manufacture and use of the composition of the invention. Since many embodiments of the invention can be made without departing from the spirit and scope of the invention, the invention resides in the claims hereinafter appended.

We claim:

1. A packaged cleaning composition concentrate comprising:
(a) a container for holding a cleaning composition concentrate; and
(b) a cleaning composition concentrate having an actives content of at least about 1 wt. % based on the weight of the cleaning composition concentrate, wherein the cleaning composition concentrate comprises:
(i) a surfactant component;
(ii) a dispersant component; and
(iii) at least one of a sheeting agent or a humectant.

2. A packaged cleaning composition concentrate according to claim 1, wherein the container comprises a film formed from at least one of polyethylene, polypropylene, polybutylene, polyester, or polyamide.

3. A packaged cleaning composition concentrate according to claim 1, wherein the container comprises a water soluble or water dispersible film.

4. A packaged cleaning composition concentrate according to claim 3, wherein the water soluble polymer or water dispersible polymer comprises at least one of polyvinyl alcohol, cellulose ethers, polyethylene oxide, starch, polyvinylpyrrolidone, polyacrylamide, polyvinyl methyl ether-maleic anhydride, polymaleic anhydride, styrene maleic anhydride, hydroxyethylcellulose, methylcellulose, polyethylene glycols, carboxymethylcellulose, polyacrylic acid salts, alginates, acrylamide copolymers, guar gum, casein, ethylene-maleic anhydride resin series, polyethyleneimine, ethyl hydroxyethylcellulose, ethyl methylcellulose, or hydroxyethyl methylcellulose.

5. A packaged cleaning composition concentrate according to claim 1, wherein in the cleaning composition concentrate is provided on a substrate.

6. A packaged cleaning composition concentrate according to claim 5, wherein the substrate comprises an opening for receiving a bottle dip tube to extend therethrough.

7. A packaged cleaning composition concentrate according to claim 1, wherein the packaged cleaning composition concentrate is provided in the form of a pouch.

8. A packaged cleaning composition concentrate according to claim 1, wherein the packaged cleaning composition concentrate is provided in the form of a capsule.

9. A packaged cleaning composition concentrate according to claim 1, wherein the packaged cleaning composition concentrate is provided in the form of a multiple reservoir cartridge configured to fit in the neck of a bottle.

10. A packaged cleaning composition concentrate according to claim 1, wherein the surfactant component comprises an anionic surfactant comprising at least one of alkyl aryl sulfonate, secondary alkane sulfonate, alkyl methyl ester sulfonate, alpha olefin sulfonate, alkyl ether sulfate, alkyl sulfate, alcohol sulfate, and mixtures thereof.

11. A packaged cleaning composition concentrate according to claim 10, wherein the cleaning composition concentrate contains about 0.1 wt. % to about 30 wt. % of the anionic surfactant component.

12. A packaged cleaning composition concentrate according to claim 1, wherein the surfactant component comprises a nonionic surfactant comprising at least one of alcohol alkoxylate, amide, polyalkylene oxide, alkyl polyglucocide, or mixture thereof.

13. A packaged cleaning composition concentrate according to claim 12, wherein the cleaning composition contains about 0.1 wt. % to about 15 wt. % of the nonionic surfactant component.

14. A packaged cleaning composition concentrate according to claim 1, wherein the dispersant component comprises at least one of a polymer and an oligomer, wherein the polymer and the oligomer contain pendant carboxylic acid groups, pendant carboxylic salt groups, or mixtures thereof.

15. A packaged cleaning composition concentrate according to claim 1, wherein the dispersant component comprises at least one of poly(acrylic acid), poly (acrylic acid/maleic acid) copolymer, poly(maleic acid/olefin) copolymer.

16. A packaged cleaning composition concentrate according to claim 1, wherein the cleaning composition concentrate comprises about 0.01 wt. % to about 10 wt. % of the dispersant.

17. A packaged cleaning composition concentrate according to claim 1, wherein the cleaning composition further comprises an amphoteric surfactant comprising at least one of betaines, imidazolines, or propinates.

18. A packaged cleaning composition concentrate according to claim 17, wherein the cleaning composition concentrate comprises about 0.01 wt. % to about 15 wt. % of the amphoteric surfactant.

19. A packaged cleaning composition concentrate according to claim 1, wherein the composition comprises the sheeting agent and wherein the sheeting agent comprises at least one of nonionic block copolymer, alcohol alkoxylate, alkyl polyglycoside, zwitterionic, and mixtures thereof.

20. A packaged cleaning composition concentrate according to claim 1, wherein the composition the humectant and wherein the humectant comprises at least one of glycerine, alkylene glycol, sorbitol, alkyl polyglycoside, polybetaine polysiloxane, and mixtures thereof.

21. A packaged cleaning composition concentrate according to claim 1, wherein the cleaning composition comprises between about 0.001 wt. % and about 10 wt. % of the sheeting agent or humectant.

22. A packaged cleaning composition concentrate according to claim 1, further comprising an organic solvent.

23. A packaged cleaning composition concentrate according to claim 22, wherein the organic solvent comprises at least one of glycol ether and derivatives of glycol ether.

24. A packaged cleaning composition concentrate according to claim 19, wherein the cleaning composition comprises about 0.1 wt. % to about 99 wt. % of the organic solvent.

25. A packaged cleaning composition concentrate according to claim 1, wherein the concentrate comprises about 0.1 wt. % to about 99 wt. % water.

26. A packaged cleaning composition concentrate according to claim 1, wherein the concentrate comprises about 30 wt. % to about 95 wt. % water.

27. A packaged cleaning composition concentrate according to claim 1, wherein the cleaning composition concentrate comprises at least about 10 wt. % actives based on the weight of the cleaning composition.

28. A method for forming a cleaning composition comprising:
combining a packaged cleaning composition concentrate with water of dilution, the packaged cleaning composition concentrate comprising:
(a) a container for holding a cleaning composition concentrate; and
(b) a cleaning composition concentrate having a solids content of at least about 1 wt. % based on the weight of the cleaning composition concentrate, wherein the cleaning composition concentrate comprises:
(i) a surfactant component;
(ii) a dispersant component; and
(iii) at least one of a sheeting agent or a humectant.

29. A packaged cleaning composition concentrate according to claim 28, wherein the container comprises a water soluble or water dispersible film.

30. A packaged cleaning composition concentrate according to claim 29, wherein the water soluble polymer or water dispersible polymer comprises at least one of polyvinyl alcohol, cellulose ethers, polyethylene oxide, starch, polyvinylpyrrolidone, polyacrylamide, polyvinyl methyl ether-maleic anhydride, polymaleic anhydride, styrene maleic anhydride, hydroxyethylcellulose, methylcellulose, polyethylene glycols, carboxymethylcellulose, polyacrylic acid salts, alginates, acrylamide copolymers, guar gum, casein, ethylene-maleic anhydride resin series, polyethyleneimine, ethyl hydroxyethylcellulose, ethyl methylcellulose, or hydroxyethyl methylcellulose.

31. A packaged cleaning composition concentrate according to claim 28, wherein the packaged cleaning composition concentrate is provided in the form of a pouch.

32. A packaged cleaning composition concentrate according to claim 28, wherein the packaged cleaning composition concentrate is provided in the form of a capsule.

33. A packaged cleaning composition concentrate according to claim 28, wherein the surfactant component comprises an anionic surfactant comprising at least one of alkyl aryl sulfonate, secondary alkane sulfonate, alkyl methyl ester sulfonate, alpha olefin sulfonate, alkyl ether sulfate, alkyl sulfate, alcohol sulfate, and mixtures thereof.

34. A packaged cleaning composition concentrate according to claim 33, wherein the cleaning composition concentrate contains about 0.1 wt. % to about 30 wt. % of the anionic surfactant component.

35. A packaged cleaning composition concentrate according to claim 28, wherein the surfactant component comprises a nonionic surfactant comprising at least one of alcohol alkoxylate, amide, polyalkylene oxide, alkyl polyglucocide, or mixture thereof.

36. A packaged cleaning composition concentrate according to claim 35, wherein the cleaning composition contains about 0.1 wt. % to about 15 wt. % of the nonionic surfactant component.

37. A packaged cleaning composition concentrate according to claim 28, wherein the dispersant component comprises at least one of a polymer and an oligomer, wherein the polymer and the oligomer contain pendant carboxylic acid groups, pendant carboxylic salt groups, or mixtures thereof.

38. A packaged cleaning composition concentrate according to claim 28, wherein the dispersant component comprises at least one of poly(acrylic acid), poly (acrylic acid/maleic acid) copolymer, poly(maleic acid/olefin) copolymer.

39. A packaged cleaning composition concentrate according to claim 28, wherein the cleaning composition concentrate comprises about 0.01 wt. % to about 10 wt. % of the dispersant.

40. A packaged cleaning composition concentrate according to claim 28, wherein the cleaning composition further comprises an amphoteric surfactant comprising at least one of betaines, imidazolines, or propinates.

41. A packaged cleaning composition concentrate according to claim 40, wherein the cleaning composition concentrate comprises about 0.01 wt. % to about 15 wt. % of the amphoteric surfactant.

42. A packaged cleaning composition concentrate according to claim 28, wherein the composition comprises the sheeting agent and wherein the sheeting agent comprises at least one of nonionic block copolymer, alcohol alkoxylate, alkyl polyglycoside, zwitterionic, and mixtures thereof.

43. A packaged cleaning composition concentrate according to claim 28, wherein the composition the humectant and wherein the humectant comprises at least one of glycerine, alkylene glycol, sorbitol, alkyl polyglycoside, polybetaine polysiloxane, and mixtures thereof.

44. A packaged cleaning composition concentrate according to claim 28, wherein the cleaning composition comprises between about 0.001 wt. % and about 10 wt. % of the sheeting agent or humectant.

45. A packaged cleaning composition concentrate according to claim 28, further comprising an organic solvent.

46. A packaged cleaning composition concentrate according to claim 45, wherein the organic solvent comprises at least one of glycol ether and derivatives of glycol ether.

47. A packaged cleaning composition concentrate according to claim 28, wherein the concentrate comprises about 30 wt. % to about 95 wt. % water.

48. A packaged cleaning composition concentrate according to claim 28, wherein the cleaning composition concentrate comprises at least about 10 wt. % actives based on the weight of the cleaning composition.

49. A method according to claim 28, wherein the water of dilution comprises water having at least 5 grains hardness.

50. A method for forming a cleaning composition in a spray bottle comprising:
placing a multiple reservoir cartridge in the neck of the spray bottle, wherein the multiple reservoir cartridge contains multiple reservoirs each containing a cleaning composition concentrate;
puncturing one of the reservoirs so that the cleaning composition concentrate combines with water of dilution in the spray bottle; wherein
the cleaning composition concentrate has a solids content of at least about 1 wt. % based on the cleaning composition concentrate, and comprises a surfactant component, a dispersant component, and at least one of a sheeting agent or humactant.

51. A method for forming a cleaning composition in a spray bottle comprising:
combining a substrate containing a cleaning composition concentrate with water of dilution in the spray bottle, wherein the cleaning composition concentrate has a solid content of at least about 1 wt. % based on the weight of the cleaning composition concentrate, and comprises a surfactant component, a dispersant component, and at least one of a sheeting agent or humactant.

52. A method according to claim 51, wherein the substrate comprises a substrate having an opening for receiving a bottle dip tube.

53. A method according to claim 52, wherein the substrate comprises a nonwoven substrate.

54. A method according to claim 51, wherein the substrate and cleaning composition concentrate are packaged in a container comprising a film formed from at least one of polyethylene, polypropylene, polybutylene, polyester, or polyamide.

55. A method for forming a cleaning composition comprising:
combining a cleaning composition concentrate with water of dilution at a weight ratio of the concentrate to the water of dilution of about 1:1 to about 1:1000 to provide a cleaning composition, wherein the cleaning composition concentrate comprises an actives level of at least about 1 wt. % and comprises about 0.1 wt. % to about 30 wt. % anionic surfactant component, about 0.01 wt. % to about 10 wt. % of a dispersant, and about 0.01 wt. % to about 10 wt. % of a sheeting agent or humactant, and wherein the water of dilution comprises water having a hardness of at least about 1 grain.

56. A method according to claim 55, wherein the method comprises a batch operation.

57. A method according to claim 55, wherein the method comprises a continuous operation wherein the cleaning composition concentrate is pumped or aspirated into a water stream comprising the water of dilution.

58. A method according to claim 57, wherein the cleaning composition concentrate comprises about 0.1 wt. % to about 10 wt. % hydrotrope.
